(19) United States
(12) Patent Application Publication
(10) Pub. No.: US 2008/0200556 A1
(43) Pub. Date: Aug. 21, 2008

(54) Amine Compounds And Inhibiting Neurotrasmitter Reuptake

(75) Inventors: Elliott Richelson, Ponte Vedra Beach, FL (US); Paul R. Carlier, Blacksburg, VA (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/570,115

(22) PCT Filed: Jun. 7, 2005
(86) PCT No.: PCT/US05/19866

§ 371 Date: Dec. 6, 2007

Related U.S. Application Data
(60) Provisional application No. 60/577,738, filed on Jun. 7, 2004.

(51) Int. Cl. A61K 31/135; C07C 215/02; A61P 25/00

(52) U.S. Cl. 514/653; 564/355

(57) ABSTRACT

The invention relates to amine compounds as well as methods and materials involved in modulating neurotransmitter reuptake. For example, the invention provides amine compounds, methods for synthesizing amine compounds, and methods for inhibiting neurotransmitter reuptake.

### BACKGROUND

1. Technical Field

The invention relates to amine compounds as well as methods and materials involved in modulating neurotransmitter reuptake.

2. Background Information

Neuronal signals are transmitted from cell to cell at specialized sites of contact known as synapses. The usual mechanism of transmission is indirect. The cells are electrically isolated from one another, the presynaptic cell being separated from the postsynaptic cell by a narrow synaptic cleft. A change of electrical potential in the presynaptic cell triggers it to release signaling molecules known as neurotransmitters. The neurotransmitters rapidly diffuse across the synaptic cleft and provoke an electrical change in the postsynaptic cell by binding to neurotransmitter receptor-gated ion channels. After release, the excess neurotransmitters are rapidly removed, either by specific enzymes in the synaptic cleft or by reuptake into the presynaptic cell or surrounding glial cells. Reuptake is mediated by a variety of neurotransmitter transporters. Rapid removal ensures both spatial and temporal precision of signaling at a synapse. For example, rapid reuptake can prevent excess neurotransmitters from influencing neighboring cells and can clear the synaptic cleft before the next pulse of neurotransmitter release so that the timing of repeated, rapid signaling events is accurately communicated to the postsynaptic cell.

An imbalance of neurotransmitters in the brain can occur when not enough neurotransmitter is made and released from presynaptic cells or the reuptake of neurotransmitters by presynaptic cells is too rapid. If neurotransmitters such as serotonin, norepinephrine, or dopamine are not made and released in effective amounts or are cleared from the synaptic cleft too quickly, then cell-to-cell communication can be affected. Clinical manifestations of such imbalances include depression and anxiety disorders. Serotonin-, norepinephrine-, dopamine-reuptake inhibitors (SNDRIs) represent a class of potent, wide-spectrum antidepressant medications that inhibit the reuptake of these neurotransmitters back into presynaptic cells. Inhibiting neurotransmitter reuptake can increase the amount of neurotransmitter present in the synapse, thus helping to normalize the transmission of neuronal signals and alleviate the symptoms of depression and anxiety disorders.

### SUMMARY

The invention relates to amine compounds as well as methods and materials involved in modulating neurotransmitter reuptake. For example, the invention provides amine compounds, methods for synthesizing amine compounds, and methods for inhibiting neurotransmitter reuptake. The amine compounds provided herein can be used as potent, wide-spectrum antidepressant medications for inhibiting neurotransmitter reuptake and treating anxiety or depressive disorders. In addition, the methods provided herein for synthesizing amine compounds allow for synthesis in a reliable and efficient manner.

In general, the invention features a composition containing N,N-dimethyl-3-cyclohexyl-3-hydroxy-2-(2′-naphthyl)propylamine. The N,N-dimethyl-3-cyclohexyl-3-hydroxy-2-(2′-naphthyl)propylamine can contain (2R, 3R)—N,N-dimethyl-3-cyclohexyl-3-hydroxy-2-(2′-naphthyl)propylamine or (2S, 3S)—N,N-dimethyl-3-cyclohexyl-3-hydroxy-2-(2′-naphthyl)propylamine. The N,N-dimethyl-3-cyclohexyl-3-hydroxy-2-(2′-naphthyl)propylamine can contain (2R,3S)—N,N-dimethyl-3-cyclohexyl-3-hydroxy-2-(2′-naphthyl)propylamine or (2S,3R)—N,N-dimethyl-3-cyclohexyl-3-hydroxy-2-(2′-naphthyl)propylamine. The N,N-dimethyl-3-cyclohexyl-3-hydroxy-2-(2′-naphthyl)propylamine can contain (a) two compounds selected from the following group: (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine; (b) three compounds selected from the following group: (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine; or (c) (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine. The composition can contain a pharmaceutically acceptable carrier. At least about 35 percent of the composition (e.g., at least about 40, 45, 50, 55, 60, 65, 70, 75, 80, 85, 90, 95, or 99 percent of the composition) can be the N,N-dimethyl-3-cyclohexyl-3-hydroxy-2-(2′-naphthyl)propylamine.

In another aspect, the invention features a method for inhibiting neurotransmitter reuptake in a mammal (e.g., human). The method includes administering a composition containing at least one compound to the mammal, wherein the composition contains at least one compound selected from the following group: N,N-dimethyl-3-cyclohexyl-3-hydroxy-2-(2′-naphthyl)propylamine, (2R,3SR)-N,N-dimethyl-3-cyclohexyl-3-hydroxy-2-(2′-naphthyl)propylamine, (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine. The neurotransmitter reuptake can be norepinephrine or epinephrine reuptake. The neurotransmitter reuptake can be dopamine reuptake or serotonin reuptake. At least about 35 percent of the composition (e.g., at least about 40, 45, 50, 55, 60, 65, 70, 75, 80, 85, 90, 95, or 99 percent of the composition) can be the compound.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagram of N,N-dimethyl-3-cyclohexyl-3-hydroxy-2-(2′-naphthyl)propylamine and its four stereoisomers.

FIG. 2 is a diagram of Compound 1 and Compound 2.

### DETAILED DESCRIPTION

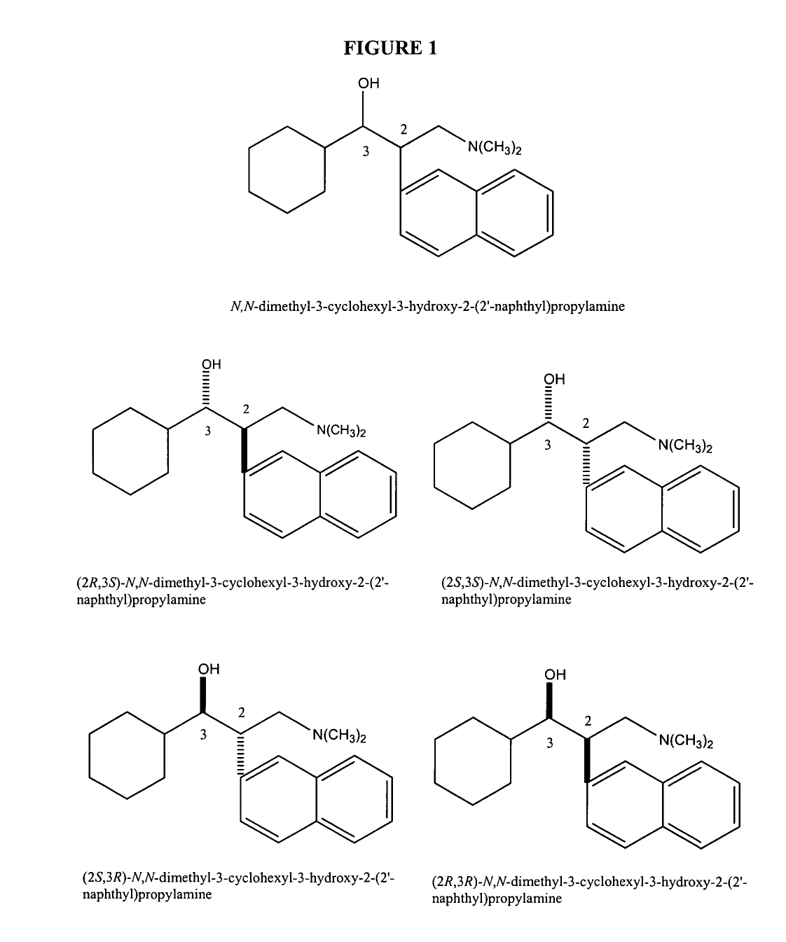

The invention relates to amine compounds as well as methods and materials involved in modulating neurotransmitter reuptake. For example, the invention provides amine compounds such as 3-hydroxy-propylamine compounds, methods for synthesizing amine compounds, and methods for inhibiting neurotransmitter reuptake. In one embodiment, the invention provides N,N-dimethyl-3-cyclohexyl-3-hydroxy-2-(2′-naphthyl)propylamine compounds (FIG. 1). It is understood that a particular 3-hydroxy-propylamine compound includes any one of that compound's stereoisomers as well as any combination thereof. For example, an N,N-dimethyl-3-cyclohexyl-3-hydroxy-2-(2′-naphthyl)propylamine compound can be (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, or (2S,3R)—N,N-dimethyl-3-cyclohexyl-3-hydroxy-2-(2′-naphthyl)propylamine, or any combination of (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine.

The invention also provides methods of synthesizing amine compounds such as 3-hydroxy-propylamine compounds. For example, 3-hydroxy-propylamine compounds can be synthesized by a variety of organic chemistry techniques including, without limitation, carbamate reduction, N,N dimethylation, aldol reduction, and nitrile reduction. An N,N-dimethyl tertiary amine compound provided herein can be synthesized by reductive methylation of a primary amine. For example, 3-cyclohexyl-3-hydroxy-2-(2′-naphthyl) propylamine can be treated with formalin, zinc chloride in methanol, and sodium cyanoborohydride to produce N,N-dimethyl-3-cyclohexyl-3-hydroxy-2-(2′-naphthyl)propylamine.

Any amine compound provided herein can be resolved to form a racemic syn-diastereomer composition, a racemic anti-diastereomer composition, or a pure enantiomer composition. For example, an amine compound can be resolved to a pure enantiomer by classical resolution using enantiomerically pure acids including, without limitation, (+)- and (−)-tartaric acid, (+)- and (−)-ditoluoyl-tartaric acid, and (+)- and (−)-camphorsulfonic acid.

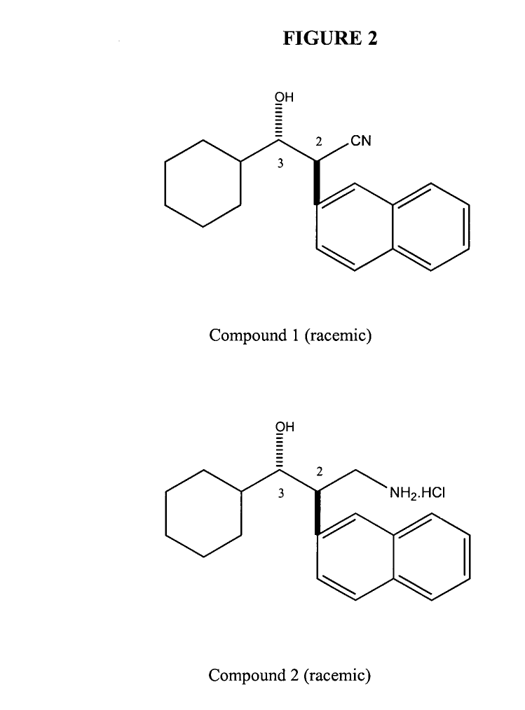

Any method can be used to isolate diastereomers and enantiomers such as those described elsewhere (Eliel et al., In: Stereochemistry of Organic Compounds; John Wiley & Sons: New York, 1994). For example, a racemic syn-diastereomer of Compound 1 (50:50 2R,3R and 2S,3S; FIG. 2) can be isolated by chromatography of the crude product mixture, or the mother liquor of the crystallization used to isolate Compound 1. Reduction of this racemic syn-isomer of Compound 1 can yield the racemic syn-diastereomer of Compound 2 (FIG. 2), which can be methylated to give the racemic syn-diastereomer of N,N-dimethyl-3-cyclohexyl-3-hydroxy-2-(2′-naphthyl)propylamine.

The racemic anti-diastereomer of N,N-dimethyl-3-cyclohexyl-3-hydroxy-2-(2′-naphthyl)propylamine (50:50 of the 2R,3S and 2S,3R enantiomers) can be resolved into the pure enantiomers by classical resolution. For example, racemic PRC025 can be treated with several different enantiomerically pure acids. The resulting amine carboxylate salts can be recrystallized. After obtaining a diastereomerically pure salt, it can be free-based, yielding enantiomerically pure PRC025.

Any amine compound or enantiomer thereof provided herein can be chemically converted from its free base form to a pharmaceutically acceptable salt by reacting the free base with an equivalent amount of any acid that forms a non-toxic salt. Such acids can be either inorganic or organic including, without limitation, hydrochloric acid, hydrobromic acid, fumaric acid, maleic acid, succinic acid, sulfuric acid, phosphoric acid, tartaric acid, acetic acid, citric acid, and oxalic acid. Any amine compound or pharmaceutically acceptable salt thereof can be administered to a mammal by itself or in combination with a carrier. Such carriers include, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Preservatives, flavorings, and other additives such as, for example, antimicrobials, anti-oxidants, chelating agents, inert gases, and the like can also be present. It will be appreciated that any amine compound provided herein that is to be administered to a mammal can contain zero, one, or more than one commonly known pharmaceutically acceptable carriers.

The invention provides methods for using amine compounds such as 3-hydroxy-propylamine compounds to inhibit neurotransmitter reuptake in a mammal. The term “inhibit” as used herein with respect to neurotransmitter reuptake refers to any reduction in neurotransmitter reuptake. For example, a reduction in neurotransmitter reuptake greater than zero percent (e.g., greater than 0.1, 0.5, 1, 2, 5, 10, 25, 50, 75, or 99 percent) is considered an inhibition of neurotransmitter reuptake. In some embodiments, a compound provided herein can inhibit neurotransmitter reuptake such that the reduction in neurotransmitter reuptake is greater than zero percent (e.g., greater than 0.1, 0.5, 1, 2, 5, 10, 25, 50, 65, 75, 85, 95, or 99 percent) as compared to untreated controls (e.g., untreated mammals or cells). Any method can be used to assess whether or not neurotransmitter reuptake has been inhibited in a mammal. Such methods can be qualitative or quantitative. An example of a qualitative method includes assessing whether or not a mammal with depression experiences loss of pleasure in daily activities, significant weight loss or gain, changes in mobility (e.g., lethargy, nervousness), feelings of worthlessness, diminished ability to concentrate, or suicidal thoughts to a lesser extent following treatment with an amine compound provided herein than the extent experienced before treatment. Alternatively, such methods can be quantitative. For example, the concentration of serotonin in a platelet sample from a mammal after treatment with an amine compound can be measured and compared to the concentration of serotonin in a platelet sample from the same mammal before treatment with that amine compound. If the concentration of serotonin after treatment is reduced compared to the concentration of serotonin before treatment, then that amine compound inhibited neurotransmitter reuptake in that mammal.

To inhibit neurotransmitter reuptake, an effective amount of any amine compound provided herein can be administered to a mammal. The term “effective” as used herein refers to any amount that induces a desired level of neurotransmitter reuptake inhibition while not inducing significant toxicity in the mammal. Such an amount can be determined using the methods and materials provided herein. An effective amount of an amine compound or formulation containing an amine compound can be any amount that reduces, prevents, or eliminates an anxiety or depressive disorder upon administration to a mammal without producing significant toxicity to that mammal. Some amine compounds may have a relatively broad concentration range that is effective while others may have a relatively narrow effective concentration range. In addition, the effective amount can vary depending upon the specific mammal or the specific anxiety or depressive disorder to be treated because certain mammals and anxiety or depressive disorders can be more or less responsive to a particular amine compound. Such effective amounts can be determined for individual amine compounds using commonly available or easily ascertainable information involving equilibrium dissociation constants, mammal toxicity concentrations, and bioavailability. For example, non-toxic amine compounds typically can be directly or indirectly administered to a mammal in any amount that reduces, prevents, or eliminates an anxiety or depressive disorder in that mammal. Using the information provided herein, such effective amounts can also be determined by routine experimentation in vitro or in vivo. For example, a patient having an anxiety or depressive disorder can receive direct administration of an amine compound in an amount close to the equilibrium dissociation constant (i.e., K_{d}) calculated from in vitro analysis sufficient to inhibit the uptake of a particular neurotransmitter. If the patient fails to respond, then the amount can be increased by, for example, two fold. After receiving this higher concentration, the patient can be monitored for both responsiveness to the treatment and toxicity symptoms, and adjustments made accordingly.

To help determine effective amounts of different amine compounds, it can be useful to refer to an effective amount equivalent based on the effective amount of a common drug used to treat anxiety or depressive disorders. For example, the direct administration of 0.30 mg/kg Prozac® (fluoxetine) daily for three weeks to a mammal can be an effective amount for treating anxiety or depressive disorders. The effects produced by this effective amount can be used as a reference point to compare the effects observed for other amine compounds used at varying concentrations. Once an equivalent effect is observed, then the specific effective amount for that particular amine compound can be determined. In this case, that particular amount would be termed a Prozac® effective amount equivalent.

The ability of an amine compound to inhibit neurotransmitter reuptake also can be assessed in vitro. For example, the level of serotonin reuptake can be determined by measuring the amount of radiolabeled serotonin taken up by synaptosomes (“pinched-off” nerve endings) purified from a tissue source abundant in serotonin transporters (e.g., rat brain cortical tissue). Rat brain cortical tissue can be isolated to produce neuronal membrane fragments such that the membrane fragments close back on themselves to form synaptosomes that retain functional serotonin transporters. The serotonin transporters concentrate serotonin by transporting it from the fluid in which the synaptosomes are suspended to the interior of the synaptosomes. If the serotonin in the suspension fluid is radiolabeled, then the level of serotonin reuptake can be measured by counting the radioactivity in the synaptosomal pellet obtained by rapid filtration or centrifugation. The ability of an amine compound to inhibit the level of serotonin reuptake can be determined by adding different concentrations to aliquots of the same synaptosomal preparation. For example, the potency of N,N-dimethyl-3-cyclohexyl-3-hydroxy-2-(2′-naphthyl)propylamine as an inhibitor of serotonin reuptake can be measured by (1) adding different concentrations of N,N-dimethyl-3-cyclohexyl-3-hydroxy-2-(2′-naphthyl)propylamine to aliquots of synaptosomes purified from rat brain cortical tissue, (2) adding the same concentration of radiolabeled serotonin to each aliquot, (3) allowing the serotonin transporters to concentrate the radiolabeled serotonin in the synaptosomes, and (4) counting the radioactivity in the synaptosomal pellet of each aliquot obtained after centrifugation. Amine compounds with a higher potency will more effectively inhibit reuptake thus resulting in less detectable radioactivity in the synaptosomal pellet.

In another in vitro example, intact cultured mammalian cells expressing a particular recombinant neurotransmitter transporter can be used to assess the ability of an amine compound to inhibit neurotransmitter reuptake. For example, the potency of N,N-dimethyl-3-cyclohexyl-3-hydroxy-2-(2′-naphthyl)propylamine as an inhibitor of norepinephrine transport can be measured using cultured mammalian cells expressing the norepinephrine transporter. In addition, the potency of a particular amine compound to inhibit multiple neurotransmitter transporters can be measured. For example, the potency of N,N-dimethyl-3-cyclohexyl-3-hydroxy-2-(2′-naphthyl)propylamine as an inhibitor of both serotonin and norepinephrine transport can be measured using separate cultured mammalian cells expressing the serotonin transporter and cultured mammalian cells expressing the norepinephrine transporter. It is understood that measured neurotransmitter transport levels are compared to controls. Controls include, without limitation, vehicle only as well as known inhibitors such as Prozac®, Paxil® (paroxetine), Effexor® (venlafaxine), or Norpramin® (desipramine).

In addition, the potency of an amine compound to inhibit the reuptake of different neurotransmitters can be assessed by determining the equilibrium dissociation constant (i.e., K_{d}) of that particular amine compound for a particular neurotransmitter transporter. Typically, the K_{d} value is determined as described elsewhere (Tatsumi et al, Euro. J. Pharmacol., 340:249-258 (1997)). Once determined, the K_{d} value for a particular amine compound can be used to compare that compound's potency with the potency of other amine compounds or other known inhibitors. For example, if a particular compound has a K_{d} of 4.1 nM for the serotonin transporter and a K_{d} of 12.5 nM for the norepinephrine transporter, then that particular compound can be characterized as having a greater ability to inhibit serotonin reuptake compared to norepinephrine reuptake. Likewise, if a first amine compound has a K_{d} of 54 nM for the dopamine transporter and a second amine compound has a K_{d} of 134 nM for the dopamine transporter, then the first amine compound can be characterized as having a greater ability to inhibit dopamine reuptake compared to the second amine compound.

Various factors can influence the actual effective amount used for a particular application. For example, the frequency of administration, duration of treatment, rate of metabolism of the drug, combination of other amine compounds, and site of administration may require an increase or decrease in the actual effective amount administered.

The frequency of administration can be any frequency that reduces, prevents, or eliminates an anxiety disorder or depression in a mammal without producing significant toxicity to the mammal. For example, the frequency of administration can be from about once a day to about once a month, or more specifically, from about twice a day to about once a week. In addition, the frequency of administration can remain constant or can be variable during the duration of treatment. As with the effective amount, various factors can influence the actual frequency of administration used for a particular application. For example, the effective amount, duration of treatment, rate of metabolism of the drug, combination of other amine compounds, and site of administration may require an increase or decrease in administration frequency.

An effective duration for amine compound administration can be any duration that reduces, prevents, or eliminates an anxiety or depressive disorder in a mammal without producing significant toxicity to the mammal. Thus, the effective duration can vary from several days to several weeks, months, or years. In general, the effective duration for the treatment of an anxiety or depressive disorder can range in duration from several days to several years. Once the amine compound administrations are stopped, however, the treated anxiety or depressive disorder may return. Thus, the effective duration for the prevention of an anxiety or depressive disorder can last in some cases for as long as the individual is alive.

Multiple factors can influence the actual effective duration used for a particular treatment or prevention regimen. For example, an effective duration can vary with the frequency of amine compound administration, effective amine compound amount, combination of multiple amine compounds, and site of administration. It is noted that diagnostic algorithm methods can be devised to determine or reflect appropriate effective doses, durations, and frequencies.

The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity. Significant toxicity can vary for each particular mammal and each particular composition.

Any combination of amine compounds can be administered to a mammal. For example, two amine compounds can be administered together to a mammal to inhibit norepinephrine reuptake in that mammal. In another example, one or more compounds that can inhibit serotonin reuptake and one or more compounds that can inhibit dopamine reuptake can be administered together to a mammal to inhibit both serotonin and dopamine reuptake in that mammal. The efficacy of such combinations can be assessed using the methods and materials provided herein.

An amine compound or combination of amine compounds can be administered to any part of a mammal's body. For example, an amine compound can be delivered to, without limitation, spinal fluid, blood, lungs, intestines, muscle tissues, skin, joints, peritoneal cavity, or brain of a mammal. In addition, an amine compound or combination of amine compounds can be administered intravenously, intraperitoneally, intramuscularly, subcutaneously, intrathecally, intracerebroventricularly, or intradermally, orally, by inhalation, or by gradual perfusion over time. The duration of treatment can be any length of time from as short as one day to as long as the life span of the mammal (e.g., many years). For example, an amine compound can be administered daily for three months or ten years. It is also noted that the frequency of treatment can be variable. For example, an amine compound can be administered once (or twice, three times, etc.) daily, weekly, monthly, or yearly.

The invention will be further described in the following examples, which do not limit the scope of the invention described in the claims.

### EXAMPLES

### Example 1

### Human Transporter Binding Studies

Human embryonic kidney (HEK-293) cells stably transfected and constitutively expressing the human norepinephrine transporter (hNET; Pacholczyk et al., Nature, 350:350-354 (1991)), the human dopamine transporter (hDAT; Pristupa et al., Mol. Pharmacol., 45:125-135 (1994)), or the human serotonin transporter (hSERT; Ramamoorthy et al., Proc. Natl. Acad. Sci. U.S. A. 90:2542-2546 (1993)) were grown and passaged in 150-mm petri dishes with 17.5 ml of Dulbecco's modified Eagle's medium (MEM; Mediatech Inc., Herndon, Va.) containing 0.1 mM non-essential amino acid solution for MEM (Mediatech Inc.), 5% (v/v) fetal clone bovine serum product (Hyclone Laboratories, Logan, Utah), and 1 U/μL penicillin/streptomycin solution (Mediatech Inc.). The cells were incubated in 10% CO₂, 90% air at 37° C. and 100% humidity. The hNET cell cultures contained 250 μg/mL geneticin sulfate. The cells were grown to 70-80% confluency prior to harvesting.

Cell membranes containing hSERT, hNET, or hDAT were prepared from the cell lines to assay ligand binding for each of the transporters. Briefly, the cell medium was removed by aspiration, and the cells were washed with 4 mL modified Puck's D1 solution (solution 1; Richelson et al. in “Methods in Neurotransmitter Receptor Analysis” Yamamura, H. I.; Enna, S. J.; Kuhar, M. J. Eds.; New York, Raven Press, 1990, pp 147-175). The washed cells were incubated for 5 minutes at 37° C. in 10 mL solution 1 containing 100 mM ethylene glycol-bis N,N,N′,N′-tetraacetic acid (EGTA). The cells were then scraped from the flask surface with a rubber spatula, placed into a centrifuge tube, and collected by centrifugation at 1000×g for 5 minutes at 4° C. The resulting supernatant was discarded, and the cell pellet was resuspended in 0.5 to 1.0 mL of the appropriate binding buffer (described below). The resuspended cell pellet was homogenized using a Polytron for 10 seconds at setting 6. The resulting homogenate was centrifuged at about 36,000×g for 10 minutes at 4° C. The supernatant was discarded, and the pellet was resuspended in the same volume of the appropriate binding buffer and centrifuged again. The supernatant was discarded, and the final pellet containing cell membranes was resuspended in the appropriate binding buffer and stored at −80° C. until use. The final protein concentration was determined by the Lowry assay using bovine serum albumin as a standard (Lowry et al., J. Biol. Chem. 193:265-275 (1951)).

Radioligand binding assays for the indicated transporters were performed as follows. To assess binding to the cloned hSERT, cells expressing hSERT were homogenized in 50 mM Tris-HCl with 120 mM NaCl and 5 mM KCl (pH 7.4). The binding reaction consisted of 30 μg cell membrane protein, 1.0 nM [³H]imipramine (imipramine hydrochloride, benzene ring-³H, specific activity 46.5 Ci/mmol; Dupont New England Nuclear, Boston, Mass.), and varying concentrations of either unlabeled imipramine or the test amine compound. A reaction to determine non-specific binding consisted of 15 μg cell membrane protein, 1.0 nM [³H]imipramine, and 1 μM final concentration of unlabeled imipramine. The reactions were incubated at 22° C. for 60 minutes. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman Instruments, Fullerton, Calif.). Radioactivity was measured with a Beckman liquid scintillation counter (LS 5000TD).

To assess binding to the cloned hNET, cells expressing hNET were homogenized in 50 mM Tris-HCl with 300 mM NaCl and 5 mM KCl (pH 7.4). The binding reaction consisted of 25 μg cell membrane protein, 0.5 nM [³H]nisoxetine (nisoxetine HCl, [N-methyl-3H], specific activity 85.0 Ci/mmol; Amersham, Arlington Hts., IL), and varying concentrations of either unlabeled nisoxetine or the test amine compound. A reaction to determine non-specific binding consisted of 25 μg cell membrane protein, 0.5 nM [³H]nisoxetine, and 1 μM final concentration of unlabeled nisoxetine. The reactions were incubated at 22° C. for 60 minutes. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman Instruments, Fullerton, Calif.). Radioactivity was measured with a Beckman liquid scintillation counter (LS 5000TD).

To assess binding to the cloned HDAT, cells expressing HDAT were homogenized in 50 mM Tris-HCl with 120 mM NaCl (pH 7.4). The binding reaction contained 30 μg cell membrane protein, 1 nM [³H]WIN35428 (WIN35428, [N-methyl-3H], specific activity 83.5 Ci/mmol; Dupont New England Nuclear, Boston, Mass.), and varying concentrations of either unlabeled WIN35428 or the test amine compound. A reaction to determine non-specific binding contained 30 μg cell membrane protein, 1 nM [³H]WIN35428, and 10 μM final concentration of unlabeled WIN35428. The reactions were incubated at 22° C. for 1 hour. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman Instruments, Fullerton, Calif.). Radioactivity was measured with a Beckman liquid scintillation counter (LS 5000TD).

Following the radioligand binding assays, the data were analyzed using the LIGAND program (Munson and Rodbard, Analyt. Biochem., 107:220-239 (1980)) to provide values for the equilibrium dissociation constants (K_{d}). The program was modified to calculate the Hill coefficient (nH). Data are presented as geometric mean ±S.E.M. of at least three independent experiments. One-component models and two-component models were compared using the root mean square error of each fit and the F test. A low K_{d} for a compound indicates strong binding to the transporter (i.e., reuptake inhibition). Compound A was made as described in U.S. Pat. No. 6,700,018. Compound PRC025 was made as described herein.

Compound PRC025 exhibited strong binding to hSERT, hNET, and hDAT. In addition, compound PRC025 exhibited greater specificity for hSERT and hDAT than that observed with compound A and was unexpectedly found to be 2.5 times more potent at hDAT than compound A. These results demonstrate that PRC025 is more balanced at inhibiting hSERT, hNET, and hDAT than is compound A. These results also demonstrate that PRC025 can inhibit hSERT, hNET, and hDAT at therapeutic dosages.

TABLE 1
Human transporter binding data.

| | Kd (nM) | Kd (nM) | Kd (nM) |
|---|---|---|---|
| | hSERT | hNET | hDAT |
| PRC025 | 4.1 | 12.5 | 54 |
| A | 6.3 | 0.3 | 134 |
| desipramine | 18 | 0.83 | 3193 |
| duloxetine | 0.22 | 4.2 | 840 |
| milnacipran | 8.8 | 85 | 70000 |
| fluoxetine | 0.81 | 240 | 3600 |
| paroxetine | 0.13 | 40 | 491 |
| reboxetine | 58 | 7.2 | 12000 |
| sertraline | 0.29 | 420 | 25 |
| venlafaxine | 8.9 | 1060 | 9300 |
| nomifensine | 1010 | 16 | 56 |

PRC025: HCl salt of (2RS,3SR)-N,N-dimethyl-3-cyclohexyl-3-hydroxy-2-(2′-naphthyl)propylamine.
A: HCl salt of (2RS,3RS)-N-methyl-3-hydroxy-2-(2′-naphthyl)-3-phenylpropylamine.

### Example 2

### Neurotransmitter Reuptake in Rat Brain Synaptosomes

Cortical, striatal, and hippocampal tissues are dissected from freshly decapitated male Sprague-Dawley rats (125-250 g; Harlan Sprague-Dawley, Indianapolis, Ind., USA). The dissected tissues are separately homogenized in 20 volumes of ice-cold 0.32 M sucrose containing 11 mM glucose (pH 7.4) in a glass Potter-Elvehjem homogenizer with Teflon® pestle (8 strokes, 900 rpm). The homogenates are centrifuged at 1,000×g for 10 minutes. The resulting supernatant is decanted and further centrifuged at 20,000×g for 20 minutes. The supernatant is discarded, and the synaptosomes contained in the pellet are gently resuspended in oxygenated incubation buffer containing 10 mM glucose, 20 mM HEPES, 145 mM NaCl, 4.5 mM KCl, 1.2 mM MgCl₂, and 1.5 mM CaCl₂ (pH 7.4). The synaptosomal protein concentration is determined by the Lowry method using bovine serum albumin as a standard (Lowry et al., J. Biol. Chem. 193:265-275 (1951)).

Synaptosomal protein (1.0-2.5 mg) is suspended 1 mL oxygenated incubation buffer containing 10 μM pargyline to inhibit monoamine oxidase activity and 0.2 mg/mL sodium ascorbate. Separate reaction tubes containing 8 nM levo-[ring-2,5,6-³H]norepinephrine ([³H]NE; 43.7 Ci/mmol; New England Nuclear, Boston, Mass.), 4 nM 5-[1,2-³H(N)]hydroxytryptamine binoxalate ([³H]5-HT; 23.4 Ci/mmol; New England Nuclear), or 2 nM [7,8-³H]dopamine (DA) ([³H]DA; 47 Ci/mmol; Amersham, Arlington Heights, Ill.) are preincubated for 5 minutes at 37° C. in a shaking water bath (80 oscillations/minute) in the presence of the test amine compound. After preincubation, the neurotransmitter reuptake reaction is initiated by the addition of synaptosomal protein. The reaction is stopped after 5 minutes by adding 4 mL ice-cold 0.9% (w/v) sodium chloride. The stopped reactions are rapidly filtered through a Whatman GF/B glass fiber filter in a 48-place Brandel cell harvester. The filters containing deposited synaptosomes are then washed with an additional 8 mL of wash buffer. The washed filters are placed in a scintillation vial containing 5 mL of Redi-Safe (Beckman Instruments, Fullerton, Calif.) and counted. Specific reuptake is calculated as the difference between the total reuptake (zero unlabelled ligand) and nonspecific reuptake (excess unlabelled ligand).

### Example 3

### Testing Amine Compounds in Animals and Humans

Test amine compounds are prepared by formulating the amine compounds with a pharmaceutically acceptable carrier such as saline. To determine the toxic levels of each amine compound, non-human mammals (e.g., mice, rats, or lower primates) are used. Briefly, 100 rats are randomly separated into five groups of 20. One group, group A, receives a placebo, while the other groups receive a particular dose of a compound, e.g., group B receives the compound at 1 μg/kg, group C receives the compound at 100 μg/kg, group D receives the compound at 10 mg/kg, and group E receives the compound at 100 mg/kg. The rats in each group are dosed according to a prescribed plan for a predetermined period of time, and toxic levels of the administered compound are determined by measuring survival or other clinical signs such as aggressiveness, irregular bleeding, and appetite. After determining the toxic level of an amine compound, tolerability and pharmacokinetic studies are performed in normal humans.

Once the toxicity, tolerability, and pharmacokinetic studies have been performed for an amine compound, 100 human subjects having an anxiety or depressive disorder are selected for clinical trials. The subjects are separated into two groups of 50. One group, group A, receives a placebo, while the other group, group B, receives the highest possible dose of a compound that has been determined not to be toxic to a mammal. The subjects in each group are dosed according to a prescribed plan for a predetermined period of time, and the ability of the administered amine compound to alleviate anxiety or depressive disorder symptoms are determined using standard clinical methods that are used to assess anxiety or depressive disorders.

### Example 4

### Synthesis of (2R,3SR)-3-cyclohexyl-3-hydroxy-2-(2′-naphthyl)proiononitrile

(2R,3SR)-3-cyclohexyl-3-hydroxy-2-(2′-naphthyl)propiononitrile (Compound 1; FIG. 2) was made as follows. 2-Naphthylacetonitrile (1.49 g, 8.93 mmol, 1 eq) and 20 mL freshly distilled THF were placed in a 250 mL round-bottomed flask. After purging with N₂, the solution was cooled to −78° C., and 2.0 M LDA solution (5.4 mL, 10.7 mmol, 1.2 eq) was added via syringe. The mixture was stirred for 30 minutes. Cyclohexanecarbaldehyde (1.3 mL, 10.7 mmol, 1.2 eq) was added via syringe, and the resulting mixture stirred for 30 minutes. Saturated NH₄Cl (12 mL) was poured into the reaction flask at −78° C. After warming to room temperature, 20 mL 1 N HCl was added, and the aqueous layer was extracted with Et₂O (4×25 mL). The combined organic extract was dried (MgSO₄), filtered, stripped, and dried in vacuo. The crude aldol obtained was then washed with pet ether/ether (4 mL/2 mL) and recrystallized twice from hot toluene. Finally, 859 mg (34%) of the pure anti-aldol was recovered.

¹H-NMR (CDCl₃): δ 1.071-1.305 (m, 6H), 1.626-2.081 (m, 6H), 3.614 (dd, J=4.0 Hz, 7.4 Hz, 1H), 4.218 (d, J=4.0 Hz, 1H), 7.388 (d, J=10.2 Hz, 1H), 7.509-7.542 (m, 2H), 7.842-7.886 (m, 4H). ¹³C-NMR (CDCl₃) analysis: δ 25.53, 25.81, 26.11, 28.34, 29.43, 41.10, 41.44, 78.10, 118.70, 125.19, 126.65, 126.81, 127.41, 127.69, 127.90, 129.12, 130.89, 132.88, 133.24. MS (CI⁺): Calcd for C₁₉H₂₁NO: 279.16, Found: 280.2 (M+H).

### Example 5

### Synthesis of (2R,3SR)-3-cyclohexyl-3-hydroxy-2-(2′-naphthyl)propylammonium chloride

(2R,3SR)-3-cyclohexyl-3-hydroxy-2-(2′-naphthyl)propylammonium chloride (Compound 2; FIG. 2) was made as follows. A 100 mL round-bottomed flask was charged with AlCl₃ (1013 mg, 7.6 mmol, 2.5 eq) and purged with N₂ for 5 minutes. A solution was prepared by adding Et₂O (20 mL), which was then cannulated to a 250 round-bottomed flask containing LiAlH₄ (327 mg, 8.6 mmol, 2.9 eq). The resulting suspension was stirred for 30 minutes at room temperature. Compound 1 (834 mg, 3.0 mmol, 1 eq) was dissolved in 5 mL THF and added via cannula over the course of 1 minute. After stirring for 3 hours at room temperature, the reaction was quenched by cautious addition of EtOAc (30 mL). After stirring for 90 minutes, 30 mL 10% H₂SO₄ was added. The aqueous layer was washed with Et₂O (2×30 mL) and then combined and made basic with excess NaOH (2 M) solution. The aqueous layer was extracted with Et₂O (8×20 mL), washed with saturated brine (20 mL), and dried (K₂CO₃). After reducing the volume to ca. 10 mL, the solution was treated with HCl gas for 1 minute, and concentrated in vacuo to afford 0.681 g (71%) of the desired γ-hydroxy amine hydrochloride salt. (white powder).

¹H-NMR (CD₃OD): δ 1.159-2.212 (m, 1H), 3.387-3.466 (m, 2H). 3.825 (dd, J=5.7 Hz, 11.7 Hz, 1H), 4.048 (dd, J=2.4 Hz, 9.3 Hz, 1H), 7.590-7.702 (m, 3H), 7.980-8.115 (m, 4H). ¹³C-NMR (CD₃OD): δ 26.38, 27.43, 27.78, 31.83, 42.04, 45.19, 48.21, 80.19, 126.96, 127.50, 127.81, 128.83, 129.02, 129.06, 130.45, 134.70, 135.43, 137.80.

### Example 6

### Synthesis of (2R,3SR)—N,N-dimethyl-3-cyclohexyl-3-hydroxy-2-(2′-naphthyl)propylammonium chloride

A 250 mL round-bottomed flask was charged with Compound 2 (200 mg, 0.6 mmol, 1 eq) and MeOH (15 mL). To this flask, a solution of NaBH₃CN (79 mg, 1.3 mmol, 2.0 eq) and ZnCl₂ (120 mg, 0.9 mmol, 1.4 eq) in MeOH (25 mL) was added. Formalin (37 wt % HCHO, 200 μL, 2.7 mmol, 4.2 eq) was then added via syringe. The reaction was stirred under nitrogen for 6 hours, at which point 75 mL NaOH were added. The aqueous layer was extracted with Et₂O (6×25 mL). The combined organic layer was then washed with saturated brine (2×25 mL), and dried (K₂CO₃). After reducing the volume to ca. 10 mL, the organic extract was treated with HCl gas for 1 minute and concentrated in vacuo to afford 217 mg (100%) of the desired N,N-dimethyl-γ-hydroxy amine hydrochloride salt. (white sticky solid)

¹H-NMR (CD₃OD): δ 1.131-1.868 (m, 11H), 3.106 (s, 6H), 3.616-3.711 (m, 2H), 3.997-4.097 (m, 2H), 7.629-7.741 (m, 3H), 8.050-8.128 (m, 4H). ¹³C-NMR (CD₃OD): δ 26.24, 27.44, 27.78, 31.82, 41.95, 44.29, 44.79, 45.38, 64.27, 80.72, 126.93, 127.68, 127.93, 128.88, 129.05, 129.10, 130.59, 134.75, 135.47, 137.18.

### Example 7

### Compound PRC025 has Antidepressant Activity

When rats are placed in a cylinder of water from which they cannot escape, they will remain immobile in the water for the majority of the test. Administration of antidepressants to rats decreases the amount of time spent immobile and increases swimming activity in the chamber (Porsolt et al., Nature, 266:730-732 (1977)). This test can be used to screen compounds for potential antidepressant activity (Cryan et al., Trends Pharmacol. Sci., 23:238-245 (2002)).

Male Sprague-Dawley rats (200-250 g) were individually placed in vertical cylinders (height 40 cm, internal diameter 19 cm) containing water (25° C.) to a level of 15 cm, as described elsewhere (Porsolt et al., Nature, 266:730-732 (1977)). Water was changed between trials, and the procedure involved a pretest and a 5-minute test separated by 24 hours. During the pretest, rats (adapted to the experimental room for no less than 1 hour) were placed in the cylinder for 15 minutes. Following this initial exposure, the rats were dried with towels and transferred to a “drying cage” situated under a warming lamp. Fifteen minutes later, rats were injected i.p. with imipramine (15 mg/kg) as a positive control, PRC025 (10 mg/kg), or saline, and returned to their home cages. The following day, rats were transferred to the experimental room and acclimated for at least 1 hour. Rats were injected with imipramine (15 mg/kg), PRC025 (10 mg/kg), or saline both 5 hours and 30 minutes before testing, and then placed in the test chambers.

A time sampling technique was used to score behavior every 5 seconds during the 5 minute test period as described elsewhere (Detke et al., Psychopharmacology (Berl), 121:66-72 (1995)). At the end of each 5-second interval, the rat's behavior was observed and scored based on the criteria described elsewhere (Porsolt et al., Nature, 266:730-732 (1977)). The rat was considered immobile when floating motionless or making only those movements necessary to keep its head above the surface of the water. Scores for each behavior (swimming or immobility) were expressed as total counts per 5-minute session. Statistical analysis was performed using ANOVA followed by the Tukey test for post-hoc comparisons.

Rats treated with PRC025 exhibited antidepressant activity that was similar to that observed in rats treated with the positive control, imipramine (Table 2). In addition, rats treated with either PRC025 or imipramine exhibited statistically more swimming behavior than rats treated with saline (Table 2).

TABLE 2
Effects of imipramine and PRC025 in the forced swim test in rats.

| | | | Time swimming | Time immobile |
|---|---|---|---|---|
| Treatment | Dose | Number | (Counts/5 min) | (Counts/5 min) |
| Saline | — | 5 | 13 ± 1 | 45 ± 3 |
| Imipramine | 15 mg/kg | 4 | 37 ± 3* | 23 ± 3* |
| PRC025 | 10 mg/kg | 4 | 30 ± 3* | 28 ± 3* |

Values represent the mean number of counts over the 5-minute period (±SEM).
*P < 0.05 vs. saline treatment.

A tail suspension test in mice similar to that described elsewhere (Steru et al., Psychopharmacology (Berl), 85:367-370 (1985)) can corroborate the results found using the forced swim test, with possible sensitivity to a broader range of antidepressants. When mice are suspended by their tail, there is an initial period of agitation, followed by immobility. This test identifies antidepressant compounds, which decrease the duration of immobility.

Male C57B1/6J mice (25-35 g) were injected i.p. with imipramine (20 mg/kg) as a positive control, PRC025 (10 mg/kg), or saline 30 minutes before testing. Mice were then individually suspended by their tails 35 cm above the tabletop using an adhesive tape placed 1 cm from the tip of the tail. Behavior was scored every 5 seconds throughout the 6-minute test as either mobile or immobile. Mice were considered immobile only when hanging passively and completely motionless. Scores for each behavior were expressed as total counts per 5-minute session. Statistical analysis was performed using ANOVA followed by the Tukey test for post-hoc comparisons.

Mice treated with PRC025 exhibited antidepressant activity (Table 3). In addition, mice treated with either PRC025 or imipramine exhibited statistically more mobile behavior than mice treated with saline (Table 3).

TABLE 3
Effects of imipramine and PRC025 in the tail suspension test in mice.

| | | | Time mobile | Time immobile |
|---|---|---|---|---|
| Treatment | Dose | Number | (Counts/6 min) | (Counts/6 min) |
| Saline | — | 5 | 33 ± 2 | 39 ± 2 |
| Imipramine | 15 mg/kg | 7 | 51 ± 4* | 21 ± 4* |
| PRC025 | 10 mg/kg | 4 | 60 ± 3* | 12 ± 3* |

Values represent the mean number of counts over the 6-minute period (±SEM).
*P < 0.05 vs. saline treatment.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising N,N-dimethyl-3-cyclohexyl-3-hydroxy-2-(2′-naphthyl)propylamine or a salt thereof.

2. The composition of claim 1, wherein said N,N-dimethyl-3-cyclohexyl-3-hydroxy-2-(2′-naphthyl)propylamine comprises (2R,3R)—N,N-dimethyl-3-cyclohexyl-3-hydroxy-2-(2′-naphthyl)propylamine or (2S,3S)—N,N-dimethyl-3-cyclohexyl-3-hydroxy-2-(2′-naphthyl)propylamine.

3. The composition of claim 1, wherein said N,N-dimethyl-3-cyclohexyl-3-hydroxy-2-(2′-naphthyl)propylamine comprises (2R,3S)—N,N-dimethyl-3-cyclohexyl-3-hydroxy-2-(2′-naphthyl)propylamine or (2S,3R)—N,N-dimethyl-3-cyclohexyl-3-hydroxy-2-(2′-naphthyl)propylamine.

4. The composition of claim 1, wherein said N,N-dimethyl-3-cyclohexyl-3-hydroxy-2-(2′-naphthyl)propylamine comprises:
a) two compounds selected from the group consisting of (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine; or
b) three compounds selected from the group consisting of (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine; or
c) (2R,3R)—N,N-dimethyl-3-cyclohexyl-3-hydroxy-2-(2′-naphthyl)propylamine, (2S,3S)—N,N-dimethyl-3-cyclohexyl-3-hydroxy-2-(2′-naphthyl)propylamine, (2R,3S)—N,N-dimethyl-3-cyclohexyl-3-hydroxy-2-(2′-naphthyl)propylamine, and (2S,3R)—N,N-dimethyl-3-cyclohexyl-3-hydroxy-2-(2′-naphthyl)propylamine.

5. The composition of claim 1, wherein said composition comprises a pharmaceutically acceptable carrier.

6. A method for inhibiting neurotransmitter reuptake in a mammal, said method comprising administering a composition comprising N,N-dimethyl-3-cyclohexyl-3-hydroxy-2-(2′-naphthyl)propylamine or a salt thereof to said mammal.

7. The method of claim 6, wherein said neurotransmitter reuptake is norepinephrine or epinephrine reuptake.

8. The method of claim 6, wherein said neurotransmitter reuptake is dopamine reuptake.

9. The method of claim 6, wherein said neurotransmitter reuptake is serotonin reuptake.

10. The method of claim 6, wherein said mammal is a human.
